(19) United States
(12) Patent Application Publication
(10) Pub. No.: US 2026/0315784 A1
(43) Pub. Date: Oct. 15, 2026

(54) ELECTROPORATION/ELECTROCHEMOTHERAPY OF HARD TO ACCESS SENSITIVE REGIONS

(71) Applicant: Mohammad Abdolahad, Tehran (IR)

(72) Inventors: Mohammad Abdolahad, Tehran (IR); Mohammad Ali Khayamian, Tehran (IR); Hamed Abadijoo, Tehran (IR); Farshid Rostami Pouria, Tehran (IR); Majid Hasanloo, Tehran (IR); Hossein Simaee, Tehran (IR); Navid Manoochehri, Tehran (IR); Seyed Mojtaba Yazdanparast, Tehran (IR); Alvand Naserghandi, Tehran (IR); Kosar Namakin, Tehran (IR); Seyed Rouhollah Miri, Tehran (IR); Habibollah Mahmoodzadeh, Tehran (IR); Sepideh Mansouri, Tehran (IR)

(73) Assignee: Nano Hesgarsazan Salamat Arya, Tehran, OT (IR)

(21) Appl. No.: 18/689,352

(22) PCT Filed: Jan. 6, 2024
(86) PCT No.: PCT/IB2024/050124

§ 371 Date: Mar. 5, 2024

(51) Int. Cl. A61N 1/04; A61N 1/02; A61N 1/32; A61N 1/36

(52) U.S. Cl. CPC A61N 1/0412; A61N 1/025; A61N 1/0476; A61N 1/327; A61N 1/36034

(57) ABSTRACT

Disclosed herein is a system for in-vivo electroporation of a plurality of target cells in a target region of a living body. The system includes a finger probe configured to transfer a pulsed electric field to the plurality of target cells, an electrical pulse generator connected to the finger probe, and a processing unit connected to the electrical pulse generator. The finger probe is attached/mounted on a subject's finger and is put in contact with the target region when the subject's finger is placed in the vicinity of the target region. The finger probe includes an electrode substrate and a plurality of electrodes attached thereon. The processing unit is configured to perform a method, including generating a pulsed electric field between at least two electrodes of the plurality of electrodes inside the target region by applying a sequence of electric voltage pulses between the at least two electrodes.

### TECHNICAL FIELD

The present disclosure generally relates to methods and apparatus for target cells' ablation and/or delivery of therapeutic agents into a target cell via an electroporation process, and particularly, to electrodes with a designed structure appropriate for applying electroporation to target cells located in sensitive regions or areas of a patient's body which are difficult to access and use thereof.

### BACKGROUND ART

Minimally invasive treatments are newly emerged methods that have recently been able to replace conventional surgical methods in some cases of cancer treatment, such as methods based on use of ultrasonic waves that destroy tumors by stimulating mechanical waves, or methods based on heat that heat a tumor at a target position and cause it to disappear. Radiofrequency (RF) methods are examples of heating methods in which high frequency electromagnetic waves are used. Each of these methods has prominent features and on the other hand has certain disadvantages, for example, heating-based methods cannot be used near main vessels due to a destruction of intercellular matrix. On the other hand, chemotherapy-based methods also cause problems for a patient. One of the biggest and most common problems in cancer chemotherapy is side effects of drugs on healthy tissues and creation of physiological problems in vital organs of body. Many cancer deaths are due to side effects of chemotherapy drugs on vital organs of body. Today, one of the most important areas of research in cancer treatment is construction of targeted drug delivery systems to tumor areas that cause minimal damage to healthy body tissues.

One of the minimally invasive newly proposed methods is treatment of cancer based on electroporation. In this method, holes are created on cancer cells by applying high voltage electrical pulses, which cause permeability of cancer cells. Holes created by electroporation may have different lifetimes based on their size, and larger holes may last longer. Pore lifetime spans from milliseconds to hours. Holes with long life-times (more than a few hours) may disrupt the tissue homeostasis and result cell death. Depending on an amount of generated electrical field and a duration of electrical pulse, the holes created on cells can be reversible, and as a result, the hole closes after the end of electrical pulse application, and finally, cells will survive. This method is used to deliver more chemotherapy drugs to tumor cells named as electrochemotherapy (ECT) since considerable enhances the efficiency of local chemotherapy. In another case, in which a created hole on cells membrane is such that the cells cannot close it, it is called irreversible electroporation and is done to directly tumor removal without mediation of chemotherapy drugs, which is also called irreversible electroporation (IRE). Depending on type of mass and its location, and many other parameters, treatment can be done in form of ECT or IRE. In this method, high voltage electrical pulses are applied to target cancer cells or cancerous tissue in a short period of time, which leads to a disruption of ionic potential balance of cell membrane; thereby, resulting in generating holes on cells and increasing permeability of cells.

Needle-based electrodes are mostly used for electroporation treatments, and many improvements and efforts have been made to fabricate and utilize electroporation devices having needle electrodes. For example, A. Westersten et al. disclosed in a US patent application numbered as US 2006/0264807 A1 a modular electrode system including a non-symmetrically arranged plurality of needle electrodes, in which a constant-current electrical pulse is applied to the plurality of needle electrodes. The modular electrode system may facilitate delivery of electrical energy to tissues in a manner that assures that the energy dose delivered lies consistently between an upper limit a lower limit; thereby, providing increased electroporation efficiencies. However, conventional electrodes that are mostly utilized in electrochemotherapy have many drawbacks and design weaknesses. For example, conventional needle electrodes cannot be used in sensitive tissues, such as vessels, nerves, intestines, etc. due to bleeding in tissues and/or perforation of vital organs. On the other hand, plate electrodes are not effective in applying electrochemotherapy stimulation due to a lack of a proper interaction surface between tissue and electrode. Moreover, in many cases, a physician is dealing with spaces where it is not possible to apply electrochemotherapy stimulation due to a lack of a proper access.

Hence, there is a need in the art to overcome the problems and drawbacks of electrodes utilizing in an electroporation-based treatment. There is a need in the art for electroporation devices having less-invasive, and preferably, non-invasive electrodes. Specifically, there is a need for a device with electrodes to apply electroporation to cells, which have a structure that minimizes a possibility of damage and bleeding to sensitive tissues (including vascular tissues, nervous tissues, etc.). Also, there is a need for electrodes that allow access to areas that cannot be reached with conventional electrodes.

### SUMMARY OF THE DISCLOSURE

This summary is intended to provide an overview of the subject matter of this patent, and is not intended to identify essential elements or key elements of the subject matter, nor is intended to be used to determine the scope of the claimed implementations. The proper scope of this patent may be ascertained from the claims set forth below in view of the detailed description below and the drawings.

In one general aspect, the present disclosure is directed to a system for in-vivo electroporation of a plurality of target cells in a target region of a living body. In an exemplary embodiment, the system may include a finger probe, an electrical pulse generator electrically connected to the finger probe, and a processing unit electrically connected to the electrical pulse generator. In an exemplary embodiment, the finger probe may be utilized to transfer a pulsed electric field to the plurality of target cells. In an exemplary embodiment, the finger probe may be attached or mounted onto a subject's finger. In an exemplary embodiment, the finger probe may be put in contact with the target region via insertion of the subject's finger into the living body in the vicinity of the target region.

In an exemplary embodiment, the finger probe may include an electrode substrate and a plurality of electrodes. In an exemplary embodiment, the electrode substrate may include a piece of a biocompatible material. In an exemplary embodiment, the electrode substrate may include a front surface and a back surface. In an exemplary embodiment, each of the front surface and the back surface may have a surface area in a range of 1 cm² to 5 cm². In an exemplary embodiment, the back surface may be attached or mounted onto the subject's finger. In an exemplary embodiment, the plurality of electrodes may be attached on the front surface of the electrode substrate. In an exemplary embodiment, each electrode of the plurality of electrodes may include a biocompatible electrically conductive sphere. In an exemplary embodiment, each electrode of the plurality of electrodes may include a biocompatible stainless steel ball with a diameter in a range of 0.1 cm to 1 cm.

In an exemplary embodiment, the electrical pulse generator may be utilized to apply a pulsed electric field between at least two electrodes of the plurality of electrodes. In an exemplary embodiment, each electrode of the at least two electrodes may be connected to a different pole of two poles of the electrical pulse generator.

In an exemplary embodiment, the processing unit may include a memory having processor-readable instructions stored therein and a processor. In an exemplary embodiment, the processor may access the memory and execute the processor-readable instructions. In an exemplary embodiment, the processor may be utilized to perform a method when the processor-readable instructions are executed by the processor. In an exemplary embodiment, the method may include inducing electroporation to the plurality of target cells by generating the pulsed electric field between the at least two electrodes of the plurality of electrodes inside the target region via applying at least one sequence of electric voltage pulses between the at least two electrodes utilizing the electrical pulse generator.

In an exemplary embodiment, applying the at least one sequence of electric voltage pulses between the at least two electrodes may include applying at least one sequence of eight square-wave electric voltage pulses with a magnitude in a range of 500 V/cm to 1500 V/cm and a duration of 100 μs between the at least two electrodes.

In an exemplary embodiment, the method may further include generating the pulsed electric field between every two electrodes of the plurality of electrodes by periodically changing a connection of the plurality of electrodes to the two poles of the electrical pulse generator. In an exemplary embodiment, periodically changing the connection of the plurality of electrodes to the two poles of the electrical pulse generator may include periodically substituting connection of at least one electrode of the at least two electrodes by a different electrode of the plurality of electrodes. In an exemplary embodiment, substituting connection of the at least one electrode of the at least two electrodes by the different electrode of the plurality of electrodes may include electrically disconnecting the at least one electrode of the at least two electrodes from a first pole of the electrical pulse generator and electrically connecting the different electrode of the plurality of electrodes to the first pole of the electrical pulse generator. In an exemplary embodiment, periodically changing the connection of the plurality of electrodes to the two poles of the electrical pulse generator may include stepwise changing the connection of the plurality of electrodes to the two poles of the electrical pulse generator during a plurality of time steps. In an exemplary embodiment, each time step of the plurality of time steps may include a time interval between 0.5 second and 5 seconds.

In an exemplary embodiment, the system may further include a couple of magnets attaching the back surface of the electrode substrate and the subject's finger together. In an exemplary embodiment, the couple of magnets may include a first magnet adhered onto the back surface of the electrode substrate and a second magnet fixed on tip of the subject's finger. In an exemplary embodiment, each of the first magnet and the second magnet may include a magnet with a surface area in a range of 0.1 cm² to 1 cm². In an exemplary embodiment, the first magnet and the second magnet are attached to each other while conducting the method. In an exemplary embodiment, the second magnet may be placed inside a glove at a respective location of the subject's finger. In an exemplary embodiment, the glove may be worn by the subject.

In an exemplary embodiment, the electrode substrate may have a thickness in a range of 0.1 cm to 1 cm. In an exemplary embodiment, a distance between each two electrodes of the plurality of electrodes may be within a range of 0.5 mm to 2 cm on the electrode substrate. In an exemplary embodiment, the plurality of electrodes may include four electrodes arranged in a square arrangement on the front surface. In an exemplary embodiment, a length of each side of the square may be in a range of 0.5 mm to 2 cm.

In an exemplary embodiment, the finger probe may be placed at a hard to access part of the living body. In an exemplary embodiment, the hard to access part of the living body may include at least one of inside esophagus, surface of tracheal, behind bladder, anus entrance, narrow spaces inside at least one of digestive, respiratory, urogenital organs, or vessels, and combinations thereof.

In an exemplary embodiment, the system may further include a plurality of electrically conductive lines, where each electrically conductive line may include a distal end and a proximal end. In an exemplary embodiment, the distal end may be connected to an electrode of the plurality of electrodes and the proximal end may be connected to a pole of the two poles of the electrical pulse generator. In an exemplary embodiment, the electrode substrate may include a plurality of holes thereon. In an exemplary embodiment, each distal end of each electrically conductive line of the plurality of electrically conductive lines may pass through a corresponding hole of the plurality of holes and attaching to a respective electrode of the plurality of electrodes.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A schematically shows an exemplary finger probe attached to an exemplary subject's finger, consistent with one or more exemplary embodiments of the present disclosure.

FIG. 1B shows a front view of an exemplary finger probe, consistent with one or more exemplary embodiments of the present disclosure.

FIG. 1C shows a back view of an exemplary finger probe, consistent with one or more exemplary embodiments of the present disclosure.

FIG. 1D shows a first exploded view of an exemplary finger probe, consistent with one or more exemplary embodiments of the present disclosure.

FIG. 1E shows a second exploded view of an exemplary finger probe, consistent with one or more exemplary embodiments of the present disclosure.

FIG. 1F shows an exemplary system for electroporation using an exemplary finger probe, consistent with one or more exemplary embodiments of the present disclosure.

FIG. 2 shows an exemplary method for electroporation of an exemplary plurality of target cells, consistent with one or more exemplary embodiments of the present disclosure.

FIG. 3 shows an example computer system in which an embodiment of the present disclosure, or portions thereof, may be implemented as computer-readable code, consistent with one or more exemplary embodiments of the present disclosure.

### DESCRIPTION OF EMBODIMENTS

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

Herein, methods, systems, and devices are disclosed for an electrical treatment (e.g., electroporation) of target cells, particularly cells of a patient's tissues. In an exemplary embodiment, methods and devices may be utilized for target cells' ablation (e.g., tumor cells destruction) and/or delivery of a substance (e.g., a drug, a diagnostic agent, a therapeutic agent, etc.) to exemplary target cells. In an exemplary embodiment, an exemplary device (e.g., a probe) may be disclosed for target cell's electroporation having one or more electrodes designed based on a location and accessibility of target cells and specific properties of exemplary target cells. In an exemplary embodiment, electrodes of an exemplary device may be designed based on sensitivity of target cells and surrounding areas to an electric field applied during an exemplary electroporation process. In an exemplary embodiment, electrodes of an exemplary probe may be non-invasive electrodes while needle electrodes of conventional electroporation devices may be invasive and unsuitable for all parts of a patient's body.

In one general embodiment of the present disclosure, a probe is disclosed. In an exemplary embodiment, an exemplary probe may be utilized by attaching to a subject's finger, named as a finger probe, herein. In an exemplary embodiment, an exemplary finger probe may be utilized for a non-invasive electrical treatment of a plurality of target cells, which may be located at a hard-to-access location in a living body of a human or an animal. In an exemplary embodiment, an exemplary finger probe may be utilized for non-invasive electrical treatment of an exemplary plurality of target cells located at areas of an exemplary living body where there is no access to exemplary areas except a surgeon's finger. In an exemplary embodiment, an exemplary finger probe may be utilized for a non-invasive electrical treatment of a plurality of target cells located at a location of at least one of inside esophagus, surface of tracheal, behind bladder, anus entrance, or narrow spaces inside at least one of digestive, respiratory, urogenital organs, or vessels, and combinations thereof. In an exemplary embodiment, an exemplary finger probe may be utilized for at least one of electrically stimulating of an exemplary plurality of target cells, electroporating an exemplary plurality of target cells, generating an electric field among an exemplary plurality of target cells, electrochemotherapy (ECT) of an exemplary plurality of target cells, electrically ablation of an exemplary plurality of target cells, and combinations thereof. In an exemplary embodiment, an exemplary finger probe may be utilized for at least one of electrically stimulating of an exemplary plurality of target cells in an area of an exemplary living body, where inserting a needle electrode there into may not be allowed or inserting a needle there may be harmful. As used herein, “target cells” may refer to cells of a part of a living body to be treated by an electrical stimulation (e.g., electroporation); allowing for treatment of exemplary cells including ablating exemplary cells and/or delivery of specific substances (e.g., a drug, a diagnostic agent, a therapeutic agent, etc.) to exemplary cells.

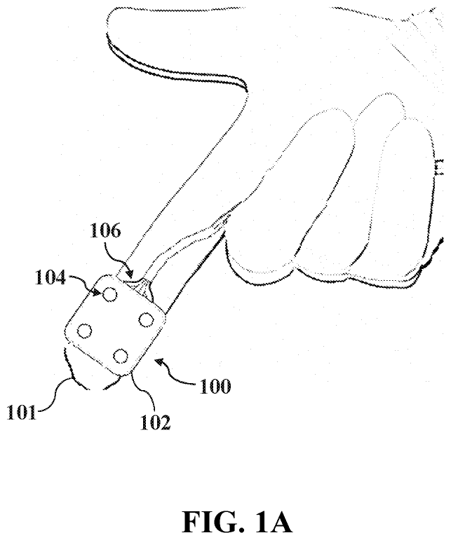

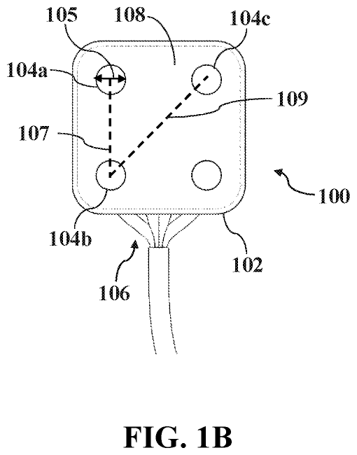

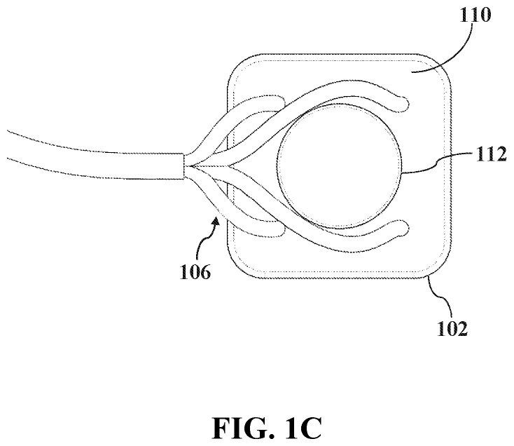

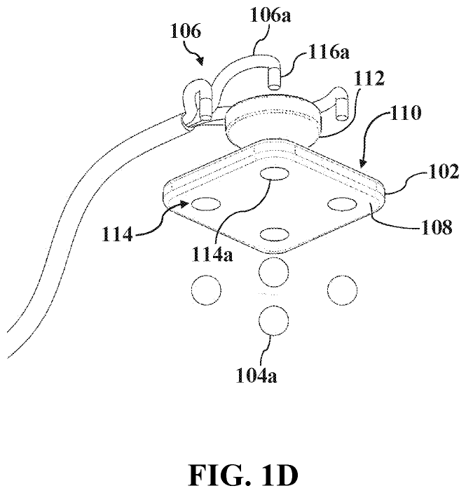

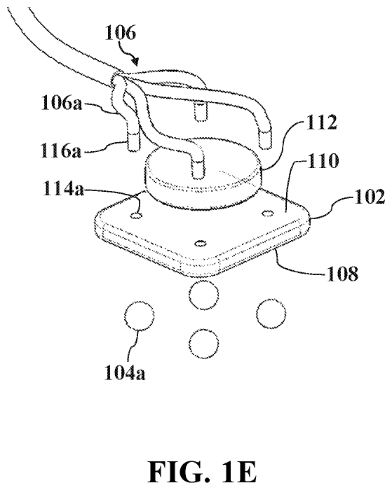

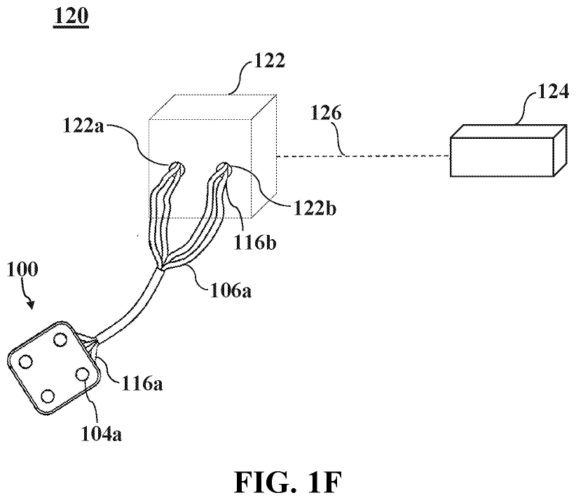

FIG. 1A schematically shows a finger probe 100 attached to a subject's finger 101, consistent with one or more exemplary embodiments of the present disclosure. In an exemplary embodiment, finger probe 100 may include an electrode substrate 102 and a plurality of electrodes 104 attached onto electrode substrate. In an exemplary embodiment, plurality of electrodes 104 may be connected to an electrical device via a respective plurality of electrically conductive lines 106. In an exemplary embodiment, an exemplary electrical device may include an electrical voltage generator or an electrical power supply. In an exemplary embodiment, an exemplary electrical device may include an electrical pulse generator. In an exemplary embodiment, electrode substrate 102 may include a piece of a biocompatible material. In an exemplary embodiment, electrode substrate 102 may include a piece of a biocompatible material with a thickness in a range of about 0.1 cm to about 1 cm. In an exemplary embodiment, electrode substrate 102 may be made of a flexible biocompatible. In an exemplary embodiment, electrode substrate 102 may be made of at least one of silicone rubber, polyethylene terephthalate (PET), polydimrthylsiloxane (PDMS), and combinations thereof.

FIGS. 1B-1E show different views of finger probe 100, consistent with one or more exemplary embodiments of the present disclosure. FIG. 1B shows a front view of finger probe 100, consistent with one or more exemplary embodiments of the present disclosure. Furthermore, FIG. 1C shows a back view of finger probe 100, consistent with one or more exemplary embodiments of the present disclosure. In an exemplary embodiment, electrode substrate 102 may include a front surface 108 and a back surface 110. In an exemplary embodiment, each of front surface 108 and back surface 110 may have a surface area in a range of about 1 cm² to about 5 cm².

In an exemplary embodiment, each electrode 104a of plurality of electrodes 104 may include a sphere-shaped electrode made of a biocompatible electrically conductive material attached onto electrode substrate 102. In an exemplary embodiment, each electrode 104a may include a biocompatible stainless steel ball. In an exemplary embodiment, each electrode 104a may have a diameter 105 in a range of about 0.1 cm to about 1 cm.

In an exemplary embodiment, plurality of electrodes 104 may be arranged in a regular or irregular arrangement. In an exemplary embodiment, plurality of electrodes 104 may be arranged in at least one of a triangular, a rectangular, a circular, a square-shaped arrangement, and combinations thereof. Referring to FIG. 1B, a distance between each two electrodes of plurality of electrodes 104 may be in a range of about 0.5 mm to about 2 cm. For example, a distance 107 between two electrodes 104a and 104b or a distance 109 between two electrodes 104b and 104c of plurality of electrodes 104 may be in a range of about 0.5 mm to about 2 cm. In an exemplary embodiment, plurality of electrodes 104 may include four electrodes arranged in a square arrangement on front surface 108 with a side in a range of about 0.5 mm to about 2 cm.

In an exemplary embodiment, plurality of electrodes 104 may be adhered, attached, or fixed on front surface 108. FIGS. 1D and 1E show two exploded views of finger probe 100, consistent with one or more exemplary embodiments of the present disclosure. In an exemplary embodiment, plurality of electrically conductive lines 106 may be utilized to fasten plurality of electrodes 104 on front surface 108 further to connect plurality of electrodes 104 to an exemplary electrical pulse generator. In an exemplary embodiment, electrode substrate 102 may include a plurality of holes 114 thereon. In an exemplary embodiment, each distal end 116a of each electrically conductive line 106a of plurality of electrically conductive lines 106 may pass through a respective hole 114a of plurality of holes 114. In an exemplary embodiment, each distal end 116a may be attached to an exemplary electrode 104a of plurality of electrodes 104; allowing for fixing and fastening electrode 104a onto front surface 108. In an exemplary embodiment, each distal end 116a may include a magnet or a ferromagnetic material attaching to electrode 104a. In an exemplary embodiment, each exemplary electrode 104a of plurality of electrodes 104 may be adhered onto front surface 108 using a paste or welding joint.

In an exemplary embodiment, an exemplary electrode 104a may be connected to distal end 116a using at least one of soldering, welding, an irreversible connection, and combinations thereof. In an exemplary embodiment, an exemplary electrode 104a may be fixed onto front surface 108 at respective hole 114a, and an exemplary connection between exemplary electrode 104a and distal end 116a may be sealed behind exemplary electrode 104a onto back surface 110.

In an exemplary embodiment, back surface 110 may be attached to subject's finger 101 or mounted on subject's finger 101. In an exemplary embodiment, plurality of electrodes 104 may be put in contact with an exemplary target region including an exemplary plurality of target cells when subject's finger 101 is placed inside or in the vicinity of an exemplary target region. In an exemplary embodiment, a coupling agent may be utilized for attaching back surface 110 to subject's finger 101. In an exemplary embodiment, an exemplary coupling agent may include a couple of magnets attaching back surface 110 to subject's finger 101. In an exemplary embodiment, finger probe 100 may further include a first magnet 112 adhered onto back surface 110 of electrode substrate 102. In an exemplary embodiment, first magnet 112 may attach to a second magnet (not illustrated) fixed onto subject's finger 101. In an exemplary embodiment, an exemplary second magnet may be placed inside a glove worn by an exemplary subject at a location of subject's finger 101. In an exemplary embodiment, each of first magnet 112 and an exemplary second magnet may include a magnet with a surface area in a range of about 0.1 cm² to about 1cm². In another exemplary embodiment, an exemplary coupling agent may include a paste adhering back surface 110 to subject's finger 101. In a further exemplary embodiment, one or more grooves (not illustrated) may be formed on back surface 110; allowing for mounting finger probe 100 subject's finger 101.

In an exemplary embodiment, finger probe 100 may be utilized for an electrical treatment of an exemplary plurality of target cells located in a target region of an exemplary living body (i.e., a human, or an animal), where an exemplary target region may be accessible only by subject's finger 101. In an exemplary embodiment, finger probe 100 may be utilized for generating an electric field inside an exemplary target region between at least two electrodes 104a and 104b; allowing for electrically stimulation of an exemplary target cells. In an exemplary embodiment, finger probe 100 may be utilized for an exemplary electrical treatment of an exemplary plurality of target cells in an exemplary target region, where an invasive insertion of a needle-like electrode may not be allowed due to a sensitiveness of an exemplary target region. Furthermore, finger probe 100 may be utilized for an exemplary electrical treatment of an exemplary plurality of target cells in an exemplary target region, where an access there into is limited and an exemplary target region may be accessible only by subject's finger 101. In an exemplary embodiment, an exemplary electrical treatment of an exemplary plurality of target cells may include in-vivo electroporation of an exemplary plurality of target cells. In an exemplary embodiment, plurality of electrodes 104 may be utilized for transferring a pulsed electric field from an exemplary electrical pulse generator to an exemplary plurality of target cells while finger probe 100 is put in contact with an exemplary target region. In an exemplary embodiment, finger probe 100 may be put in contact with an exemplary target region via insertion of subject's finger 101 into an exemplary living body in the vicinity of an exemplary target region. In an exemplary embodiment, finger probe 100 may be placed at a hard to access part of an exemplary living body, where an exemplary hard to access part of an exemplary living body may include at least one of inside esophagus, surface of tracheal, behind bladder, anus entrance, narrow spaces inside at least one of digestive, respiratory, urogenital organs, or vessels, and combinations thereof.

In an exemplary embodiment, plurality of electrodes 104 may be utilized for transferring (applying) an electrical signal (e.g., a pulsed electric field) to an exemplary plurality of target cells to be treated via electrical stimulation (e.g., electroporation). In an exemplary embodiment, plurality of electrodes 104 may be put in contact with a zone of at least one of a tissue, an organ, or a portion thereof including an exemplary plurality of target cells. In an exemplary embodiment, an electric potential may be applied between at least two electrodes of plurality of electrodes 104 so that an exemplary electric field may be generated in an area including an exemplary plurality of target cells. In an exemplary embodiment, a pulsed electric field may be applied between at least two electrodes of plurality of electrodes 104 and an exemplary pulsed electric field may be generated inside an exemplary zone including an exemplary plurality of target cells. In an exemplary embodiment, an exemplary plurality of target cells of at least one of an exemplary tissue, an exemplary organ, or an exemplary portion thereof may be affected by an exemplary electric field applied between at least two electrodes of plurality of electrodes 104. In an exemplary embodiment, an exemplary plurality of target cells may be electroporated due to an exemplary electric field applied between at least two electrodes of plurality of electrodes 104.

In an exemplary embodiment, an exemplary zone of at least one of a tissue, an organ, or a portion thereof including an exemplary plurality of target cells may be swept and scanned by moving subject's finger 101 all over an exemplary zone and applying an exemplary electric field; thereby, resulting in electrically stimulating (e.g., electroporating) of all exemplary plurality of target cells. In an exemplary embodiment, a contact between plurality of electrodes 104 and an exemplary zone including an exemplary plurality of target cells may not need an insertion of plurality of electrodes 104 into an exemplary zone and a superficial contact between plurality of electrodes 104 and an exemplary zone may be effective for generating an exemplary electric field in an exemplary zone and electrically affecting exemplary plurality of target cells so that a completely non-invasive contact between plurality of electrodes 104 and an exemplary zone, and consequently, a completely non-invasive electrically stimulation of an exemplary plurality of target cells may be achieved. In an exemplary embodiment, plurality of electrodes 104 may be put over skin at a location adjacent to an exemplary zone. In another exemplary embodiment, plurality of electrodes 104 may be put inside a person's body at a location adjacent to an exemplary zone while a direct access to an exemplary zone being provided during a surgery.

In an exemplary embodiment, finger probe 100 may be utilized for applying a therapeutic method via electrical stimulation (e.g., electroporation) of an exemplary plurality of target cells of an exemplary living body, for example, tumor cells of a cancer patient. In an exemplary embodiment, a system for electroporation including finger probe 100 may be disclosed. FIG. 1F shows a system 120 for electroporation using finger probe 100, consistent with one or more exemplary embodiments of the present disclosure. In an exemplary embodiment, system 120 may include finger probe 100, an electrical pulse generator 122, and a processing unit 124.

In an exemplary embodiment, electrical pulse generator 122 may be electrically connected to finger probe 100 via connecting line 140. In an exemplary embodiment, electrical pulse generator 122 may be an example of an exemplary power supply to be utilized for applying an electrical field between at least two electrodes of plurality of electrodes 104 of finger probe 100. In an exemplary embodiment, electrical pulse generator 122 may include an electroporation pulse generator. In an exemplary embodiment, electrical pulse generator 122 may be electrically connected to plurality of electrodes 104 of finger probe 100 via plurality of plurality of electrically conductive lines 106. In an exemplary embodiment, a distal end 116a of each electrically conductive line 106a may be connected to an exemplary electrode 104a of plurality of electrodes 104 and a proximal end 116b of each electrically conductive line 106a may be connected to electrical pulse generator 122. In an exemplary embodiment, electrical pulse generator 122 may be utilized to apply electrical pulses between one or more pairs of plurality of electrodes 104 while plurality of electrodes 104 being put in contact with an exemplary zone in a living body including an exemplary plurality of target cells; thereby, resulting in generating a pulsed electric field within an exemplary zone stimulating an exemplary plurality of target cells.

Referring to FIG. 1F, processing unit 124 may be electrically connected to electrical pulse generator 122 via a wireless connection or utilizing respective electrically conductive wire 126. In an exemplary embodiment, processing unit 124 may include a memory having processor-readable instructions stored therein and a processor. In an exemplary embodiment, an exemplary processor may be utilized to access an exemplary memory and execute exemplary processor-readable instructions. In an exemplary embodiment, executing exemplary processor-readable instructions by an exemplary processor may configure an exemplary processor to perform a method. In an exemplary embodiment, an exemplary method may include applying a therapeutical treatment to an exemplary plurality of target cells inside an exemplary living body by electrically stimulating an exemplary plurality of target cells. In an exemplary embodiment, an exemplary method may include at least one of treating an exemplary plurality of target cells, ablating an exemplary plurality of target cells, delivering a drug or therapeutical substance to an exemplary plurality of target cells, and combinations thereof by electroporation an exemplary plurality of target cells.

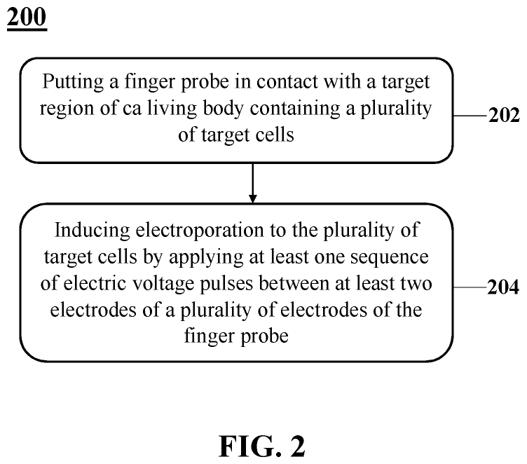

FIG. 2 shows a method 200 for electroporation of an exemplary plurality of target cells, consistent with one or more exemplary embodiments of the present disclosure. In an exemplary embodiment, method 200 may include putting finger probe 100 in contact with an exemplary target region of an exemplary living body containing an exemplary plurality of target cells (step 202) and inducing electroporation to an exemplary plurality of target cells by generating an exemplary pulsed electric field between at least two electrodes of plurality of electrodes 104 of finger probe 100 inside an exemplary target region (step 204). In an exemplary embodiment, method 200 may be carried out utilizing probe 100 and system 120. So, method 200 may be described herein below in connection with FIGS. 1A-1F.

In further detail with respect to step 202, finger probe 100 may be put in contact with an exemplary target region of an exemplary living body, where an exemplary target region may contain an exemplary plurality of target cells. In an exemplary embodiment, putting finger probe 100 in contact with an exemplary target region of an exemplary living body may include inserting finger probe 100 into an exemplary living body in the vicinity of an exemplary target region. In an exemplary embodiment, putting finger probe 100 in contact with an exemplary target region of an exemplary living body may include placing tip of a finger of a subject (e.g., a surgeon) at which finger probe 100 may be mounted or attached. In an exemplary embodiment, putting finger probe 100 in contact with an exemplary target region of an exemplary living body may include placing an exemplary tip of an exemplary finger of an exemplary subject at a location of at least one of inside esophagus, surface of tracheal, behind bladder, anus entrance, a narrow space inside at least one of digestive, respiratory, urogenital organs, or vessels, and combinations thereof. In an exemplary embodiment, putting finger probe 100 in contact with an exemplary target region may include placing finger probe 100 on exemplary plurality of target cells to be treated. In an exemplary embodiment, putting finger probe 100 in contact with an exemplary target region may include sweeping or scanning an exemplary target region by finger probe 100 via moving an exemplary finger of an exemplary subject inside/on an exemplary target region while conducting step 204 of method 200.

In an exemplary embodiment, in many cases of sarcoma or carcinoma, a small, sensitive to invasive insertion of electrodes, and/or hard to access part of an exemplary living body may include cancer cells to be electroporated. In some cases, inserting an electroporation electrode of common electroporation devices may not be possible due to a very narrow or small space of an exemplary target region. Furthermore, inserting of needle-like electrodes may cause damage or bleeding in an exemplary sensitive target region. In such cases, finger probe 100 may be put in contact with an exemplary target region with no invasive insertion or any limitations about accessibility to an exemplary target region.

In further detail with respect to step 204, step 204 may include inducing electroporation to an exemplary plurality of target cells by generating an exemplary pulsed electric field between at least two electrodes of plurality of electrodes 104 of finger probe 100 inside an exemplary target region. In an exemplary embodiment, step 204 may include inducing electroporation to an exemplary plurality of target cells by generating an exemplary pulsed electric field between at least two electrodes of plurality of electrodes 104 of finger probe 100 inside an exemplary target region via applying at least one sequence of electric voltage pulses between exemplary at least two electrodes utilizing electrical pulse generator 122. For example, step 204 may include applying at least one sequence of electric voltage pulses between at least two exemplary electrodes 104a and 104b. In an exemplary embodiment, applying at least one sequence of electric voltage pulses between at least two exemplary electrodes 104a and 104b may include applying at least one sequence of eight square-wave electric voltage pulses with a magnitude in a range of about 500 V/cm to about 1500 V/cm and a duration of about 100 μs between at least two exemplary electrodes 104a and 104b. In an exemplary embodiment, applying at least one sequence of electric voltage pulses between at least two exemplary electrodes 104a and 104b may include applying at least one sequence of eight square-wave electric voltage pulses with a magnitude of about 1000 V/cm and a duration of about 100 μs between at least two exemplary electrodes 104a and 104b.

In an exemplary embodiment, step 204 may further include generating an exemplary pulsed electric field all over an exemplary target region; allowing for electroporation of all exemplary target cells therein. In an exemplary embodiment, step 204 may further include generating an exemplary pulsed electric field between every two electrodes of plurality of electrodes 104 by generating an exemplary pulsed electric field between every possible pair of electrodes of plurality of electrodes 104. In an exemplary embodiment, generating an exemplary pulsed electric field between every two electrodes of plurality of electrodes 104 may include generating an exemplary pulsed electric field in a plurality of directions in an exemplary target region; thereby, resulting in electrical stimulation of all parts of an exemplary target region.

In an exemplary embodiment, generating an exemplary pulsed electric field between every two electrodes of plurality of electrodes 104 may include periodically changing a connection of plurality of electrodes 104 to two poles 122a and 122b of electrical pulse generator 122. In an exemplary embodiment, periodically changing an exemplary connection of plurality of electrodes 104 to two poles 122a and 122b of electrical pulse generator 122 may include periodically substituting connection of at least one electrode of at least two electrodes 104a and 104b by a different electrode 104c of plurality of electrodes 104. In an exemplary embodiment, substituting connection of at least one electrode of at least two electrodes 104a and 104b by different electrode 104c of plurality of electrodes 104 may include electrically disconnecting an exemplary at least one electrode (e.g., 104a) of at least two electrodes 104a and 104b from a first pole 122a of electrical pulse generator 122 and electrically connecting different electrode 104c of plurality of electrodes 104 to first pole 122a of electrical pulse generator 122.

In an exemplary embodiment, an exemplary connection of plurality of electrodes 104 to two poles 122a and 122b of electrical pulse generator 122 may include stepwise changing an exemplary connection of plurality of electrodes 104 to two poles 122a and 122b of electrical pulse generator 122 during a plurality of time steps. In an exemplary embodiment, each time step of an exemplary plurality of time steps may include a time interval between about 0.5 second and about 5 seconds.

In an exemplary embodiment, method 200 may be performed for a pre-determined period of time until a therapeutical target, for example, complete destruction of an exemplary plurality of cells including a plurality of cancer cells may be achieved. In an exemplary embodiment, an exemplary pre-determined period of time may depend on an exemplary therapeutical target. In an exemplary embodiment, an exemplary pre-determined period of time may depend on at least one of a location of an exemplary target region, a number of plurality of electrodes 104, an intensity of an exemplary generated pulsed electric field among plurality of electrodes 104, and combinations thereof. In an exemplary embodiment, an exemplary pre-determined period of time may include a continuous time interval or a plurality of intermittent time intervals. In an exemplary embodiment, an exemplary pre-determined period of time may include an exemplary plurality of time steps. For instance, for ECT, treatment duration cannot be more than about 20 minutes in each treatment session due to a decrease in concentration of an applied chemotherapeutic in whole bloodstream.

In an exemplary embodiment, method 200 may further include a step of injecting a drug or a therapeutical substance from a reservoir embedded in finger probe 100 or using an injection syringe into a location in the vicinity of an exemplary zone containing an exemplary plurality of cells. In an exemplary embodiment, an exemplary injected drug or therapeutical substance may penetrate into electroporated exemplary plurality of target cells; thereby, resulting in treating an exemplary plurality of target cells.

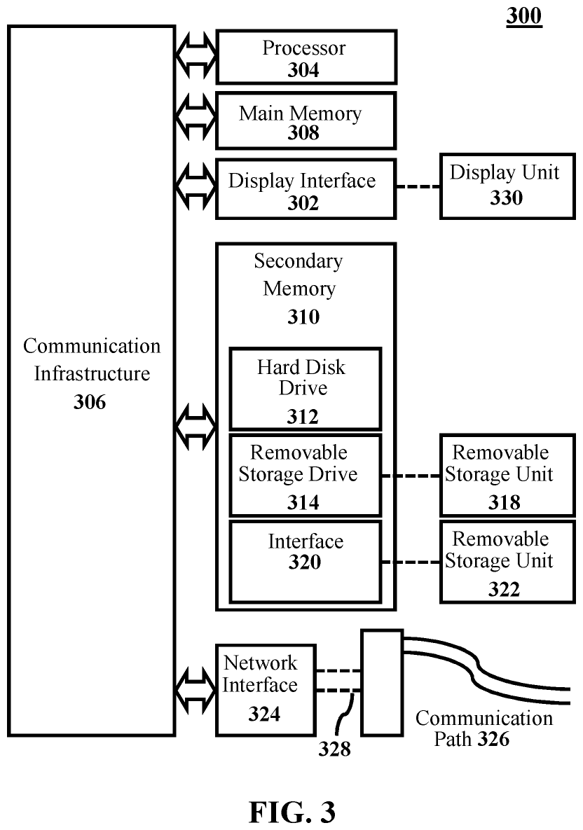

FIG. 3 shows an example computer system 300 in which an embodiment of the present disclosure, or portions thereof, may be implemented as computer-readable code, consistent with one or more exemplary embodiments of the present disclosure. For example, computer system 300 may include an example of processing unit 124, and step 204 of flowchart presented in FIG. 2 may be implemented in computer system 300 using hardware, software, firmware, tangible computer readable media having instructions stored thereon, or a combination thereof and may be implemented in one or more computer systems or other processing systems. Hardware, software, or any combination of such may embody any of the modules and components in FIGS. 1F and 2.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores”.

An embodiment of the present disclosure is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 304 may be a special purpose or a general-purpose processor device. As will be appreciated by persons skilled in the relevant art, processor device 304 may also be a single processor in a multi-core/multiprocessor system, such system operating alone, or in a cluster of computing devices operating in a cluster or server farm. Processor device 304 may be connected to a communication infrastructure 306, for example, a bus, message queue, network, or multi-core message-passing scheme.

In an exemplary embodiment, computer system 300 may include a display interface 302, for example a video connector, to transfer data to a display unit 330, for example, a monitor. Computer system 300 may also include a main memory 308, for example, random access memory (RAM), and may also include a secondary memory 310. Secondary memory 310 may include, for example, a hard disk drive 312, and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer usable storage medium having stored therein computer software and/or data.

In alternative embodiments, secondary memory 310 may include other similar means for allowing computer programs or other instructions to be loaded into computer system 300. Such means may include, for example, a removable storage unit 322 and an interface 320. Examples of such means may include a program cartridge and cartridge interface (such as that found in video game devices), a removable memory chip (such as an EPROM, or PROM) and associated socket, and other removable storage units 322 and interfaces 320 which allow software and data to be transferred from removable storage unit 322 to computer system 300.

Computer system 300 may also include a communications interface 324. Communications interface 324 allows software and data to be transferred between computer system 300 and external devices. Communications interface 324 may include a modem, a network interface (such as an Ethernet card), a communications port, a PCMCIA slot and card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g. DRAMs, etc.).

Computer programs (also called computer control logic) are stored in main memory 508 and/or secondary memory 310. Computer programs may also be received via communications interface 324. Such computer programs, when executed, enable computer system 300 to implement different embodiments of the present disclosure as discussed herein. In particular, the computer programs, when executed, enable processor device 304 to implement the processes of the present disclosure, such as the operations in method 200 illustrated by FIG. 2, discussed above. Accordingly, such computer programs represent controllers of computer system 300. Where an exemplary embodiment of method 200 is implemented using software, the software may be stored in a computer program product and loaded into computer system 300 using removable storage drive 314, interface 320, and hard disk drive 312, or communications interface 324.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### INDUSTRIAL APPLICABILITY

Disclosed herein are devices for in-vivo electroporation of target cells in sensitive and/or difficult-to-access regions of a living body as well as systems and methods utilizing thereof. An exemplary device includes non-invasive structured electrodes capable of being placed in a hard to access target region or in the vicinity of the target region without causing bleeding, tearing, or any injuries. An exemplary device includes a finger probe fixed on a subject's finger (e.g., a surgeon's finger), and limited areas of a living body can be exposed to a pulsed electric field applied to electrodes of an exemplary finger probe by inserting or putting the subject's finger at/inside an exemplary target region.

For example, in some cancer cases, tissue involvement occurs in areas where no access to these areas is possible using any electrode (due to limited space). Herein, design of an exemplary finger probe makes it possible for exemplary electrodes to be effectively used in these cases. For example, in esophageal cancers, when the esophagus is released from the trachea and it is known that cancer has transferred from radial wall of the esophagus to the trachea and it is possible that it is infected on surface of the trachea, there is no access to the infected site in this area except a surgeon's finger. So, an appropriate way for electrochemotherapy in these areas is to use an exemplary finger probe. Another example is sarcoma or carcinoma that affects back of the bladder, which is adjacent to the abdominal area. Electrochemotherapy in this area is not possible using conventional methods because it is not possible to enter any electrode in this small space. Using an exemplary finger probe is a solution for electrochemotherapy of this area, because several fingers can be placed directly behind the bladder. In this case, back of the bladder can be scanned using a finger electrode and electrochemotherapy treatment can be applied. Another example is involvement of the anus in patients with rectal cancer. If there is a possibility of involvement of the rectum in the entrance of the anus and the lumen of this area, an exemplary finger probe can be used for electrochemotherapy of this area.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A system for in-vivo electroporation of a plurality of target cells in a target region of a living body, the system comprising:
a finger probe configured to transfer a pulsed electric field to the plurality of target cells, the finger probe being attached or mounted onto a subject's finger and being put in contact with the target region via insertion of the subject's finger into the living body in the vicinity of the target region, the finger probe comprising:
an electrode substrate, comprising a piece of a biocompatible material, the electrode substrate comprising a front surface and a back surface, each with a surface area in a range of 1 cm² to 5 cm², the back surface being attached or mounted onto the subject's finger; and
a plurality of electrodes attached on the front surface of the electrode substrate, each respective electrode comprising a biocompatible electrically conductive sphere with a diameter in a range of 0.1 cm to 1 cm;
an electrical pulse generator configured to apply a pulsed electric field between at least two electrodes of the plurality of electrodes, each electrode of the at least two electrodes being connected to a different pole of two poles of the electrical pulse generator; and
a processing unit electrically connected to the electrical pulse generator, the processing unit comprising:
a memory having processor-readable instructions stored therein; and
a processor configured to access the memory and execute the processor-readable instructions, which, when executed by the processor configures the processor to perform a method, the method comprising:
inducing electroporation to the plurality of target cells by generating the pulsed electric field between the at least two electrodes of the plurality of electrodes inside the target region via applying at least one sequence of electric voltage pulses between the at least two electrodes utilizing the electrical pulse generator.

2. The system of claim 1, wherein applying the at least one sequence of electric voltage pulses between the at least two electrodes comprises applying at least one sequence of eight square-wave electric voltage pulses with a magnitude in a range of 500 V/cm to 1500 V/cm and a duration of 100 μs between the at least two electrodes.

3. The system of claim 1, wherein the method further comprises generating the pulsed electric field between every two electrodes of the plurality of electrodes by periodically changing a connection of the plurality of electrodes to the two poles of the electrical pulse generator.

4. The system of claim 3, wherein periodically changing the connection of the plurality of electrodes to the two poles of the electrical pulse generator comprises periodically substituting connection of at least one electrode of the at least two electrodes by a different electrode of the plurality of electrodes, substituting connection of the at least one electrode of the at least two electrodes by the different electrode of the plurality of electrodes comprises:
electrically disconnecting the at least one electrode of the at least two electrodes from a first pole of the electrical pulse generator; and
electrically connecting the different electrode of the plurality of electrodes to the first pole of the electrical pulse generator.

5. The system of claim 3, wherein periodically changing the connection of the plurality of electrodes to the two poles of the electrical pulse generator comprises stepwise changing the connection of the plurality of electrodes to the two poles of the electrical pulse generator during a plurality of time steps.

6. The system of claim 5, wherein each time step of the plurality of time steps comprises a time interval between 0.5 second and 5 seconds.

7. The system of claim 1, further comprising a couple of magnets attaching the back surface of the electrode substrate and the subject's finger together, the couple of magnets comprising:
a first magnet adhered onto the back surface of the electrode substrate, the first magnet comprising a magnet with a surface area in a range of 0.1 cm² to 1 cm²; and
a second magnet fixed on tip of the subject's finger, the second magnet comprising a magnet with a surface area in a range of 0.1 cm² to 1 cm²,
wherein the first magnet and the second magnet are attached to each other while conducting the method.

8. The system of claim 1, wherein a distance between each two electrodes of the plurality of electrodes is within a range of 0.5 mm to 2 cm on the electrode substrate.

9. The system of claim 1, wherein the finger probe is placed at a hard to access part of the living body, the hard to access part of the living body comprising at least one of inside esophagus, surface of tracheal, behind bladder, anus entrance, narrow spaces inside at least one of digestive, respiratory, urogenital organs, or vessels, and combinations thereof.

10. The system of claim 1, further comprising a plurality of electrically conductive lines, each respective electrically conductive line comprising a distal end and a proximal end, the distal end being connected to an electrode of the plurality of electrodes, the proximal end being connected to a pole of the two poles of the electrical pulse generator.

11. The system of claim 1, wherein the plurality of electrodes comprises four electrodes arranged in a square arrangement on the front surface, a length of each side of the square being in a range of 0.5 mm to 2 cm.

12. The system of claim 1, wherein the electrode substrate comprises a flexible biocompatible material, the flexible biocompatible material comprising at least one of silicone rubber, polyethylene terephthalate (PET), polydimethylsiloxane (PDMS), and combinations thereof.

13. A finger probe for in-vivo electroporation of a plurality of target cells in a target region of a living body, the finger probe comprising:
an electrode substrate, comprising a piece of a biocompatible material, the electrode substrate comprising a front surface and a back surface, each with a surface area in a range of 1 cm² to 5 cm², the back surface being attached to the subject's finger; and
a plurality of electrodes attached on the front surface of the electrode substrate, each respective electrode comprising a biocompatible electrically conductive sphere with a diameter in a range of 0.1 cm to 1 cm, the plurality of electrodes configured to transfer a pulsed electric field to the plurality of target cells while the finger probe being put in contact with the target region via insertion of the subject's finger into the living body in the vicinity of the target region,
wherein the finger probe is configured to be placed at a hard to access part of the living body, the hard to access part of the living body comprising at least one of inside esophagus, surface of tracheal, behind bladder, anus entrance, narrow spaces inside at least one of digestive, respiratory, urogenital organs, or vessels, and combinations thereof.

14. The finger probe of claim 13, wherein the electrode substrate has a thickness in a range of 0.1 cm to 1 cm.

15. The finger probe of claim 13, further comprising a first magnet adhered onto the back surface of the electrode substrate, the first magnet configured to attach to a second magnet fixed onto the subject's finger, each of the first magnet and the second magnet comprising a magnet with a surface area in a range of 0.1 cm² to 1 cm².

16. The finger probe of claim 15, wherein the second magnet is placed inside a glove at a respective location of the subject's finger, the glove configured to be worn by the subject.

17. The finger probe of claim 13, wherein a distance between each two electrodes of the plurality of electrodes is within a range of 0.5 mm to 2 cm.

18. The finger probe of claim 13, further comprising a plurality of electrically conductive lines, each respective electrically conductive line comprising a distal end and a proximal end, the distal end being connected to an electrode of the plurality of electrodes, the proximal end being connected to a pole of the two poles of an electrical pulse generator.

19. The finger probe of claim 18, wherein the electrode substrate comprises a plurality of holes thereon, each respective distal end of each electrically conductive line of the plurality of electrically conductive lines passing through a respective hole of the plurality of holes and attaching to a respective electrode of the plurality of electrodes.

20. The finger probe of claim 13, wherein the plurality of electrodes comprises four electrodes arranged in a square arrangement on the front surface, a length of each side of the square being in a range of 0.5 mm to 2 cm.
